(19) United States
(12) Patent Application Publication
(10) Pub. No.: US 2024/0307004 A1
(43) Pub. Date: Sep. 19, 2024

(54) SYSTEM AND METHOD FOR MONITORING HEALTH PARAMETERS WITH MATCHED DATA

(71) Applicant: KNOW LABS, INC., Seattle, WA (US)

(72) Inventor: JOHN CRONIN, Seattle, WA (US)

(21) Appl. No.: 18/603,947

(22) Filed: Mar. 13, 2024

Related U.S. Application Data
(60) Provisional application No. 63/452,783, filed on Mar. 17, 2023.

(51) Int. Cl. A61B 5/00; A61B 5/05; A61B 5/145

(52) U.S. Cl. CPC A61B 5/7267; A61B 5/05; A61B 5/14532; A61B 5/7246; A61B 5/7278

(57) ABSTRACT

Radio frequency scanning data is generated by transmitting radio waves into a person and receiving radio waves, including a responded portion of the transmitted radio waves. Real-time features can be extracted from a pulse wave signal of the radio frequency scanning data. The extracted real-time features can be matched to waveforms of a standard extracted feature waveform database to generate matched data. Features can be extracted from at least one of the pulse-wave signals, and a mathematical model can be generated in response to the pulse-wave signal. A machine learning engine, including a trained model, can process the extracted features and the matched data and output a health parameter of the person.

### FIELD

The present disclosure is generally related to systems and methods of monitoring health parameters and, more particularly, relates to a system and a method of monitoring real-time glucose levels using RF Activated-range radio frequency signals.

### BACKGROUND

The subject matter discussed in the background section should not be assumed to be prior art merely due to its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

Diabetes is a medical condition in which a person's blood glucose level, also known as blood sugar level, is persistently elevated. Diabetes can result in severe medical complications, including cardiovascular disease, kidney disease, stroke, foot ulcers, and eye damage if left untreated. Typically, diabetes is caused by either insufficient insulin production by the pancreas, referred to as “Type 1 diabetes,” or improper insulin response by the body's cells referred to as “Type 2 diabetes.” Further, monitoring a person's blood glucose level and administering insulin when a person's blood glucose level is too high to reach the desired level may be part of managing diabetes. Depending on many factors, such as the severity of diabetes and the individual's medical history, a person may need to measure their blood glucose level up to ten times per day. Each year, billions of dollars are spent on equipment and supplies for monitoring blood glucose levels.

Moreover, regular glucose monitoring is a crucial component of diabetes care. The majority of glucose monitoring methods and devices require a blood sample. Clinical measurement of blood glucose is generally invasive, including giving a blood sample at a clinic or hospital. Home glucose monitoring is also possible using a variety of devices. With in-home devices, a blood sample is typically obtained by pricking the skin using a tiny instrument. A glucose meter or glucometer is a tiny instrument that measures the sugar in the blood sample.

Currently, available glucose monitoring devices also require a blood sample, usually by pricking a needle under the skin and then using a polling technique to determine the glucose level of a patient. These monitoring devices are almost 95 percent accurate and are also preferable by urban citizens. However, such monitoring devices are often prone to contamination as the patient may not be in sanitary conditions to give the blood sample.

### SUMMARY

A system and method to monitor glucose levels with enhanced accuracy and without requiring a blood sample from the patient. Radio frequency scanning data is generated by transmitting radio waves into a person and receiving radio waves, including a responded portion of the transmitted radio waves. Real-time features can be extracted from a pulse wave signal of the radio frequency scanning data. The extracted real-time features can be matched to waveforms of a standard extracted feature waveform database to generate matched data. Features can be extracted from at least one of the pulse-wave signals, and a mathematical model can be generated in response to the pulse-wave signal. A machine learning engine, including a trained model, can process the extracted features and the matched data and output a health parameter of the person.

In an embodiment, a method for monitoring a health parameter of a person can include receiving a pulse wave signal that is generated from radio frequency scanning data that corresponds to a response to RF Activated frequency radio waves transmitted into the person, wherein the radio frequency scanning data is collected through a two-dimensional array of receive antennas over a range of radio frequencies; inputting a standard extracted features waveform database, the standard extracted waveform database including waveforms and health parameter labels; extracting real-time features from the pulse wave signal; matching the extracted real-time features to waveforms of the standard extracted features waveform database, thereby creating matched data; extracting modeled features from a mathematical model generated in response to the pulse wave signal, wherein the mathematical model is a statistical combination of the pulse-wave signal and at least one waveform of the standard extracted waveform database; applying the extracted modeled features and the matched data to a machine-learning engine that includes a trained model; and outputting from the machine learning engine an indication of a health parameter of the person in response to the extracted features.

In another embodiment, a heath parameter monitoring system can include a device that includes one or more transmit antennas configured to transmit radio frequency (RF) waves from one or more transmit antennas into a person over a range of frequencies, and one or more receive antennas that receive return RF waves resulting from the transmitted RF waves into the person and from which a pulse wave signal is generated; a memory that stores the pulse wave signal; a standard waveform database stored in the memory; an input waveform module in communication with the memory that is configured to extracting a segment of the pulse wave signal to generate an extracted segment; a matching module in communication with the memory and that is configured to receive the extracted segment, compare the extracted segment to the waveforms in the standard waveform database thereby creating matched data, assign a correlation coefficient to each matched data, and determine which correlation coefficients exceed a threshold value; and a machine learning module with a machine learning algorithm that is configured to input into the machine learning algorithm matching waveforms from the matching module for correlation coefficients that exceed the threshold value. In an embodiment, instead of generating an extracted segment, the entire pulse wave signal is compared to the waveforms in the standard waveform database to create the matched data.

In another embodiment, a heath parameter monitoring method can include transmitting radio frequency (RF) waves from one or more transmit antennas into a person over a range of frequencies, and receiving, using one or more receive antennas, a pulse wave signal that results from the RF waves transmitted into the person; extracting a segment of the pulse wave signal to generate an extracted segment; comparing the extracted segment to waveforms in a standard waveform database thereby creating matched data; assigning a correlation coefficient to each matched data, and determining which correlation coefficients exceed a threshold value; for the correlation coefficients that exceed the threshold value, sending matching waveforms from the correlation coefficients that exceed the threshold value to a machine learning module and inputting the matching waveforms into a machine learning algorithm. In an embodiment, instead of generating an extracted segment, the entire pulse wave signal is compared to the waveforms in the standard waveform database to create the matched data.

### DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. In some examples, one element may be designed as multiple elements, or those multiple elements may be designed as one element. In some examples, an element shown as an internal component of one element may be implemented as an external component in another and vice versa. Furthermore, elements may not be drawn to scale. Non-limiting and non-exhaustive descriptions are described concerning the following drawings. The components in the figures are not necessarily to scale, emphasis instead being placed upon illustrating principles.

FIG. 1: Illustrates a device for radio frequency health monitoring according to an embodiment and a subject.

FIG. 2: Illustrates a process of operation of a Device Base Module, according to an embodiment.

FIG. 3: Illustrates a process of operation of an Input Waveform Module, according to an embodiment.

FIG. 4: Illustrates a process of operation of a Matching Module, according to an embodiment.

FIG. 5: Illustrates a process of operation of a Machine Learning Module, according to an embodiment.

FIG. 6: Illustrates a process of operation of a Notification Module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

Modules may be software and/or hardware, providing a combination of executable elements.

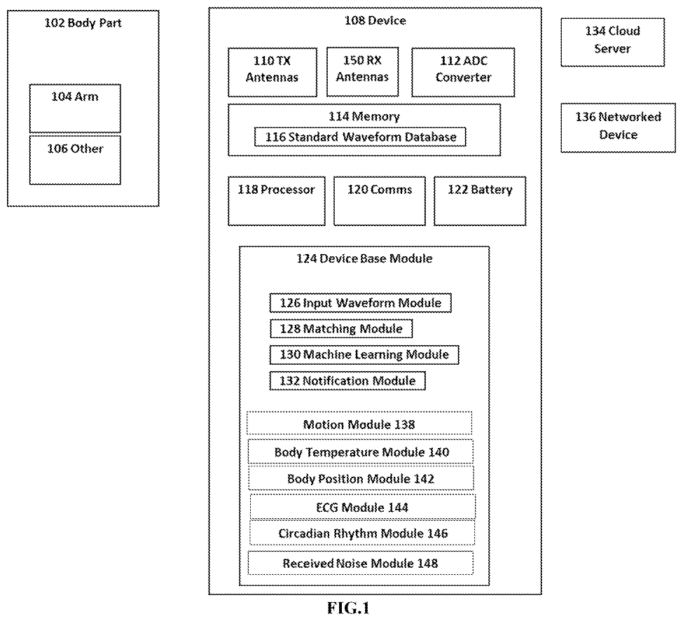

FIG. 1 shows an embodiment of a device for radio frequency health monitoring and a subject. This system comprises a device 108 attached or in proximity to a body part 102 of the subject. The body part 102 may be an arm 104. The body part 102 may be another body part 106 besides an arm, such as a leg, finger, chest, head, or any other body part from which useful medical parameters can be taken. Device 108 may be a wearable and portable device such as, but not limited to, a cell phone, a smartwatch, a tracker, a wearable monitor, a wristband, and a personal blood monitoring device.

The device 108 may further comprise a set of TX antennas 110 and RX antennas 150. TX antennas 110 may be configured to transmit RF Activated range radio frequency signals at one or more pre-defined frequencies. The RF Activated range is between 500 MHZ and 300 GHZ. In one embodiment, the pre-defined frequencies may correspond to a range a portion within the RF Activated range. For example, the one or more TX antennas 110 transmit signals selected from Activated range radio frequency signals in a range of 120-126 GHz. Successively, the one or more RX antennas 150 may be configured to receive the responded portion of the RF Activated range radio frequency signals.

The system may further comprise an ADC converter 112, which may be configured to convert the RF Activated range radio frequency signals from an analog signal into a digital processor readable format. The system may further comprise memory 114, which may be configured to store the transmitted RF Activated range radio frequency signals by the one or more TX antennas 110 and receive a responded portion of the transmitted RF Activated range radio frequency signals from the one or more RX antennas 150. Further, the memory 114 may also store the converted digital processor readable format by the ADC converter 112. The Memory 114 may include suitable logic, circuitry, and/or interfaces that may be configured to store a machine code and/or a computer program with at least one code section executable by the processor 118. Examples of implementation of the memory 114 may include, but are not limited to, Random Access Memory (RAM), Read Only Memory (ROM), Hard Disk Drive (HDD), and/or a Secure Digital (SD) card. The device base module 124 may be within memory 114 or be within another memory (not shown).

The system may further comprise a standard waveform database 116, which may contain standard waveforms for known patterns. The standard waveform database 116 may be located in memory 114 or accessible in the memory of another device. The standard waveforms of database 116 may be raw or converted device readings from patients or persons with known conditions. For example, the standard waveform database 116 may include raw or converted readings from one or more patients. In an embodiment, the readings can be from one or more patients having specific characteristics and/or measurement locations, for example reading from a right arm of a patient known to have diabetes or an average of multiple such patients, or the like. In another example, metadata such as labels for a particular measurement of glucose can be associated with the waveforms. For example, glucose waveforms taken from a patients arm just after lunch may include metadata labels such as “left arm”, “post-meal”, the patient's name and/or ID, and/or any diagnosis that may put the data in context such as diabetes or liver disease. These labels may be used to give context to doctors and organize waveform data. In another example, there may be data related to the strength of the waveform to be matched. For instance, if a waveform can be highly correlated, that information could be obtained in the matching module. This data can be compared to readings from a person with an unknown condition to determine if the waveforms from that person match any of the known standard waveforms.

The system may further comprise a processor 118, which may facilitate the operation of the device 108 according to the instructions stored in the memory 114. The processor 118 may include suitable logic, circuitry, interfaces, and/or code that may be configured to execute a set of instructions stored in the memory 114.

The system may further comprise comms 120, which may communicate with a network (not shown). Examples of networks with which comms 120 may communicate can include but are not limited to, the Internet, a cloud network, a Wireless Fidelity (Wi-Fi) network, a Wireless Local Area Network (WLAN), a Local Area Network (LAN), a telephone line (POTS), Long Term Evolution (LTE), and/or a Metropolitan Area Network (MAN).

The system may further comprise a battery 122, which may power hardware modules of the device 108. The device 108 may be configured with a charging port to recharge the battery 122. Charging of the battery 122 may be wired or wireless.

The system may further comprise a device base module 124, which may be configured to store instructions for executing the computer program from the converted digital processor readable format of the ADC converter 112. The device base module 124 may be configured to facilitate the operation of the processor 118, the memory 114, the TX antennas 110, the RD antennas 150, and the comms 120. Further, the device base module 124 may be configured to create polling of the RF Activated range radio frequency signals. It can be noted that the device base module 124 may be configured to filter the RF Activated range radio frequency signals received from one or more RX antennas 150.

The system may further comprise an input waveform module 126, which may extract a radio frequency waveform from memory. This may be the raw or converted recording of data from the RX antennas 150 from a patient wearing the device. If the entire radio frequency is too long for effective matching, the input waveform module 126 may select a time interval within the data set. This input waveform may then be sent to matching module 128. The matching module 128 is configured to match the input waveform or selected time interval thereof and each of the standard waveforms in the standard waveform database 116 by performing a convolution and/or cross-correlation of the input waveform and the standard waveform. These convolutions and/or cross-correlations can then be sent to the machine learning module 130.

The system may further comprise a machine learning module 130 which has been trained to identify health parameters based on the convolution and/or cross-correlations of the input and standard waveforms. The machine learning module 130 can receive the convolutions and cross-correlations results from the matching module 128 and outputs any health parameters identified.

The system may further comprise a notification module 132, which may determine if any of the health parameters output by the machine learning module 130 require a notification. If so, the patient and/or the patient's medical care providers may be notified.

In some embodiments, the device base module 124 may optionally utilize a motion module 138 that includes at least one sensor from the group of an accelerometer, a gyroscope, an inertial movement sensor, or other similar sensor. The motion module 138 may have its own processor or utilize the processor 118 to calculate the user's movement. Motion from the user will change the blood volume in a given portion of their body and the blood flow rate in their circulatory system. This may cause noise, artifacts, or other errors in the real-time signals received by the RX antennas 150. The motion module 138 may compare the calculated motion to a motion threshold stored in memory 114. For example, the motion threshold could be movement of more than two centimeters in one second. The motion threshold could be near zero to ensure the user is stationary when measuring to ensure the least noise in the RF signal data. When calculated motion levels exceed the motion threshold, the motion module 138 may flag the RF signals collected at the time stamp corresponding to the motion as potentially inaccurate. In some embodiments, the motion module 138 may compare RF signal data to motion data over time to improve the accuracy of the motion threshold. The motion module 138 may alert the user, such as with an audible beep or warning or a text message or alert to a connected mobile device. The alert would signal the user that they are moving too much to get an accurate measurement. The motion module 138 may update the standard waveform database 116 with the calculated motion of the user that corresponds with the received RF signal data. In this manner, the motion module 138 may be simplified to just collect motion data and allow the device base module 124 to determine if the amount of motion calculated exceeds a threshold that would indicate the received RF signal data is too noisy to be relied upon for a blood glucose measurement.

The device base module 124 may optionally utilize a body temperature module 140 that includes at least one sensor from the group of a thermometer, a platinum resistance thermometer (PRT), a thermistor, a thermocouple, or another temperature sensor. The body temperature module 140 may have its own processor or utilize the processor 118 to calculate the temperature of the user or the user's environment. The user's body temperature, the environmental temperature, and the difference between the two will change the blood volume in a given part of their body and the blood flow rate in their circulatory system. Variations in temperature from the normal body temperature or room temperature may cause noise, artifacts, or other errors in the real-time signals received by the RX antennas 150. The body temperature module 140 may compare the measured temperature to a threshold temperature stored in memory 114. For example, the environmental temperature threshold may be set at zero degrees Celsius because low temperatures can cause a temporary narrowing of blood vessels which may increase the user's blood pressure. When the measured temperature exceeds the threshold, the body temperature module 140 may flag the RF signals collected at the time stamp corresponding to the temperature as potentially being inaccurate. In some embodiments, the body temperature module 140 may compare RF signal data to temperature data over time to improve the accuracy of the temperature threshold. The body temperature module 140 may alert the user, such as with an audible beep or warning or a text message or alert to a connected mobile device. The alert would signal to the user that their body temperature, or the environmental temperature is not conducive to getting an accurate measurement. The body temperature module 140 can update the standard waveform database 116 with the measured user or environmental temperature that corresponds with the received RF signal data. In this manner, the body temperature module 140 may be simplified to just collect temperature data and allow the device base module 124 to determine if the temperature measure exceeds a threshold that would indicate the received RF signal data is too noisy to be relied upon for a blood glucose measurement.

The device base module 124 may optionally utilize a body position module 142 that includes at least one sensor from the group of an accelerometer, a gyroscope, an inertial movement sensor, or another similar sensor. The body position module 142 may have its own processor or utilize the processor 118 to estimate the user's position. The user's body position may change the blood volume in a given part of their body and the blood flow rate in their circulatory system. This may cause noise, artifacts, or other errors in the real-time signals received by the RX antennas 150. The body position module 142 may compare the estimated position to a body position threshold stored in memory 114. For example, the monitoring device 102 may be on the user's wrist, and the body position threshold may be based on the relative position of the user's hand to their heart. When a user's hand is lower than their heart, their blood pressure will increase, with this effect being more pronounced the longer the position is maintained. Conversely, the higher a user holds their arm above their heart, the lower the blood pressure in their hand. The body position threshold may include some minimum amount of time the estimated body position occurs. When the estimated position exceeds the threshold, the body position module 142 may flag the RF signals collected at the time stamp corresponding to the body position as potentially being inaccurate. In some embodiments, the body position module 142 may compare RF signal data to motion data over time to improve the accuracy of the body position threshold. The body position data may also be used to estimate variations in parameters such as blood pressure that corresponds to the body position data to improve the accuracy of the measurements taken when the user is in that position. The body position module 142 may alert the user, such as with an audible beep or warning or a text message or alert to a connected mobile device. The alert would signal to the user that their body position is not conducive to getting an accurate measurement. The body position module 142 may update the standard waveform database 116 with the estimated body position data that corresponds with the received RF signal data. In this manner, the body temperature module 140 may be simplified to just collect temperature data and allow the device base module 124 to determine if the body position exceeded a threshold that would indicate the received RF signal data is too noisy to be relied upon for a blood glucose measurement.

The device base module 124 may optionally utilize a ECG module 144 that includes at least one electrocardiogram sensor. The ECG module 144 may have its own processor or utilize the processor 118 to record the electrical signals that correspond with the user's heartbeat. The user's heartbeat will impact blood flow. Measuring the ECG data may allow the received RF data to be associated with peak and minimum cardiac output so as to create a pulse waveform allowing for the estimation of blood volume at a given point in the wave of ECG data. Variations in blood volume may cause noise, artifacts, or other errors in the real-time signals received by the RX antennas 150. The ECG module 144 may compare the measured cardiac data to a threshold stored in memory 114. For example, the threshold may be a pulse above 160 bpm, as the increased blood flow volume may cause too much noise in the received RF signal data to accurately measure the blood glucose. When the ECG data exceeds the threshold, the ECG module 144 may flag the RF signals collected at the time stamp corresponding to the ECG data as potentially being inaccurate. In some embodiments, the ECG module 144 may compare RF signal data to ECG data over time to improve the accuracy of the ECG data threshold or to improve the measurement of glucose at a given point in the cycle between peak and minimum cardiac output. The ECG module 144 may alert the user, such as with an audible beep or warning or a text message or alert to a connected mobile device. The alert would signal to the user that their heart rate is not conducive to getting an accurate measurement or requires additional medical intervention. The ECG module 144 may update the standard waveform database 116 with the measured ECG data that corresponds with the received RF signal data. In this manner, the ECG module 144 may be simplified to just collect ECG data and allow the device base module 124 to determine if the ECG data exceeded a threshold that would indicate the received RF signal data is too noisy to be relied upon for a blood glucose measurement.

The device base module 124 may optionally utilize a circadian rhythm module 146 that includes at least one sensor measuring actigraphy, wrist temperature, light exposure, and heart rate. The circadian rhythm module 146 may have its own processor or utilize the processor 118 to calculate the user's circadian health. Blood pressure follows a circadian rhythm in that it increases upon waking in the morning and decreases during sleeping at night. People with poor circadian health will often have higher blood pressure. These variations in blood pressure can cause noise, artifacts, or other errors or inaccuracies in the real-time signals received by the RX antennas 150. The circadian rhythm module 146 may compare the circadian data to a threshold stored in memory 114. For example, the threshold may be less than 6 hours of sleep in the last 24 hours. When the observed circadian health data exceeds the threshold, the circadian rhythm module 146 may flag the RF signals collected at the time stamp corresponding to circadian health as potentially being inaccurate or needing an adjustment to account for the expected increase in the user's blood pressure. In some embodiments, the circadian rhythm module 146 may compare RF signal data to sleep data over time to improve the accuracy of the circadian rhythm thresholds. The circadian rhythm module 146 may alert the user, such as with an audible beep or warning, or a text message or alert to a connected mobile device. The alert would signal to the user that their recent sleep patterns are not conducive to getting an accurate measurement. The circadian rhythm module 146 may update the standard waveform database 116 with the measured circadian data that corresponds with the received RF signal data. In this manner, the circadian rhythm module 146 may be simplified to just collect circadian rhythm data and allow the device base module 124 to determine if the measure exceeded a threshold that would indicate the received RF signal data is too noisy to be relied upon for a blood glucose measurement, or if an alternative transfer function should be used to compensate for the detected circadian health.

The device base module 124 may include a received noise module 148 that includes at least one sensor measuring background signals such as RF signals, Wi-Fi, and other electromagnetic signals that could interfere with the signals received by the RX antennas 150. The received noise module 148 may have its own processor or utilize the processor 118 to calculate the level of background noise being received. Background noise may interfere with or cause noise, artifacts, or other errors or inaccuracies in the real-time signals received by the RX antennas 150. The received noise module 148 may compare the level and type of background noise to a threshold stored in memory 114. The threshold may be in terms of field strength (volts per meter and ampere per meter) or power density (watts per square meter). For example, the threshold may be RF radiation greater than 300 μW/m2. When the background noise data exceeds the threshold, the received noise module 148 may flag the RF signals collected at the time stamp corresponding to background noise levels as potentially being inaccurate. In some embodiments, the received noise module 148 may compare RF signal data to background noise over time to improve the accuracy of the noise thresholds. The received radiation module may alert the user, such as with an audible beep or warning, a text message, or an alert to a connected mobile device. The alert would signal to the user that the current level of background noise is not conducive to getting an accurate measurement. The received noise module 148 may update the standard waveform database 116 with the background noise data that corresponds with the received RF signal data. In this manner, the received noise module 148 may be simplified to just collect background noise data and allow the device base module 124 to determine if the measure exceeded a threshold that would indicate the received RF signal data is too noisy to be relied upon for a blood glucose measurement, or if an alternative transfer function should be used to compensate for the noise.

In embodiments, one or more of memory 114, standard waveform database 116, input waveform module 126, matching module 128, the machine learning module 130, the notification module 132, the motion module 138, the body temperature module 140, the body position module 142, the ECG module 144, the circadian rhythm module 146, and/or the received noise module 148 can be provided on one or more separate devices, such as cloud server 134, the networked device 136, or the like. In such embodiments, the comms 120 can be used to communicate with the cloud server 134 or the networked device 136 to access the memory 114, standard waveform database 116, input waveform module 126, matching module 128, the machine learning module 130, the notification module 132, the motion module 138, the body temperature module 140, the body position module 142, the ECG module 144, the circadian rhythm module 146, and/or the received noise module 148 by way of any suitable network.

FIG. 2 displays an operation process for the device base module 124. The process begins with the device base module 124 polling the RF Activated range radio frequency signals between the one or more TX antennas 110 and the one or more RX antennas 150 at step 200. For example, the polling rate is used as a duty cycle of how much data is collected. In another example, if data is collected in error, the polling is reinitiated. The device base module 124 may be configured to read and process instructions stored in the memory 114 using the processor 118. For example, the device base module 124 sends signals selected from RF Activated range radio signals of frequency range 120-126 GHz to a TX antenna 110 and stores the signals selected from RF Activated range radio signals into the memory 114. The TX antenna 110 sends the signals selected from RF Activated range radio signals into the patient. The device base module 124 may receive the RF Activated range radio frequency signals from the one or more RX antennas 150 at step 202. For example, an RX antenna 150 receives a reflected RF Activated radio signal of frequency range 100-110 GHz from the patient. The device base module 124 may be configured to convert the received RF Activated range radio frequency signals into a digital format using the ADC 112 at step 204. For example, the received signals from the Activated radio signal of frequency range 100-110 GHz are converted into a 10-bit data signal. The device base module 124 may be configured to store the RF Activated range radio frequency signals that have been converted to the digital format in the memory 114 at step 206. The device base module 124 may be configured to filter the stored RF Activated range radio frequency signals at step 208. The device base module 124 may be configured to filter each RF Activated range radio frequency signal using the low pass filter. For example, the device base module 124 can filter stored signals in the RF Activated radio range or a portion thereof, for example from between 100-110 GHz that are responsive to the transmitted RF Activated range radio signals of frequency range 122-126 GHz. The device base module 124 may be configured to transmit the filtered RF Activated range radio frequency signals to the cloud server 134 or the networked device 136 using the comms module 120 at step 210. For example, the device base module 124 transmits signals selected from the Activated radio signal of frequency range 122-126 GHz to the cloud server 134. The device base module 124 may be configured to determine whether the transmitted data is already available in the cloud server 134 or another networked device 136 at step 212. The device base module 124, using the comms 120, communicates with the cloud server 134 to determine that the transmitted RF Activated radio signal of frequency range 122-126 GHz is already available. The device base module 124 may determine that the transmitted data is not already present in the cloud server 134. The device base module 124 may be redirected back to step 200 to poll the RF Activated range radio frequency signals between the one or more TX antennas 110 and the one or more RX antennas 150. For example, the device base module 124 determines that the transmitted signals selected from the RF Activated radio signal of frequency range 122-126 GHz are not present in the cloud server 134, and corresponding to the transmitted signal, there is no data related to the blood glucose level of the patient. The device base module 124 may determine that transmitted data is already present in the cloud server 134. For example, the device base module 124 can read a notification from the cloud server 134 of the patient's blood glucose level as 110 mg/dL corresponding to signals selected from the RF Activated radio signal of frequency range 122-126 GHz. The device base module 124 may continue to step 214 when it is determined that the transmitted data is present in the cloud server 134. The device base module 124 may notify the user via the device 108 of health information, for example, the blood glucose level.

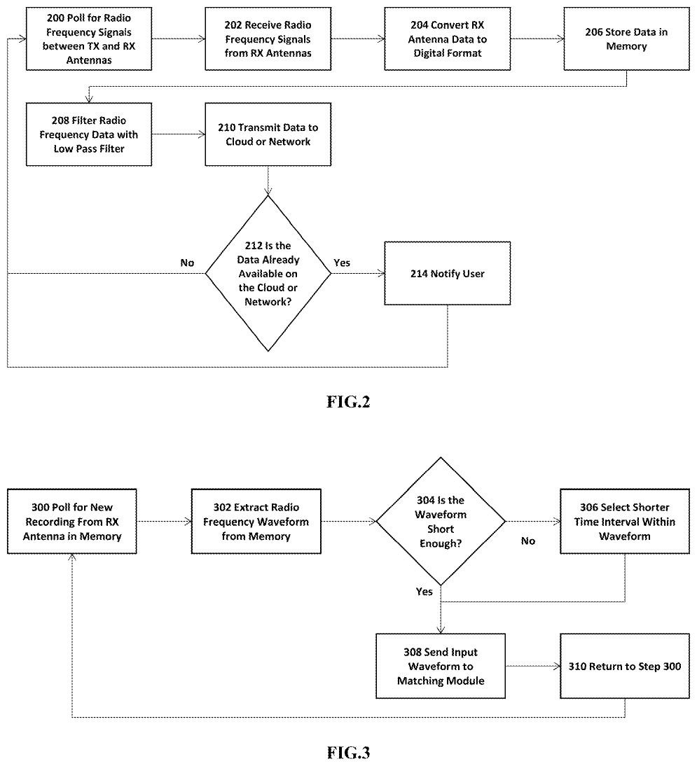

FIG. 3 displays a process for the operation of the input waveform module 126. The process may begin with the input waveform module 126 polling, at step 300, for newly recorded data from the RX antennas 150 stored in memory 114. The input waveform module 126 may extract, at step 302, the recorded radio frequency waveform from the memory 114. If there is more than one waveform recorded, the input waveform module 126 may select each waveform separately and loop through the following steps. The input waveform module 126 may determine, at step 304, if the waveform is small enough to be an input waveform for the matching module 128. This will depend on the computational requirements and/or restrictions of the matching module 128. If the waveform is short enough, the input waveform module 126 may skip to step 308. If the waveform is too long, the input waveform module 126 may select, at step 306, a shorter time interval within the entire recorded waveform. For example, if the waveform is 5 minutes long, then only a 30-second interval may be selected. The interval may be selected at random or by a selection process. For example, a selection process could be a process that selects two or more random intervals. In another example, the selection process could be a structured selection of being selected every one-fifth of the time of the time of the signal. The input waveform module 126 may send, at step 308, the input waveform to the Matching module 128. The input waveform module 126 may return, at step 310, to step 300.

FIG. 4 displays a process for the operation of the matching module 128. The process may begin with the matching module 128 polling, at step 400, for an input waveform from the input waveform module 126. The matching module 128 may extract, at step 402, some or all of the standard waveforms from the standard waveform database 116. The matching module 128 may match, at step 404, the input waveform with each standard waveform. Matching may determine which standard waveforms the input waveform is similar to. A correlation coefficient can be determined between the input waveform and each standard waveform. The correlation coefficient can be used to determine which standard waveforms are similar when the correlation coefficient exceeds a threshold value. For example, when the correlation coefficient of a standard waveform to the input waveform is >0.9, the standard waveform can be deemed to be similar to the input waveform. The threshold value for which correlation coefficient value is considered matching may be set by an administrator of the system, a user of the system, and/or automatically by the system. Matching may involve convolution and/or cross-correlation of the waveforms. Once the match is made, we can use these matched waveforms for the next steps. The matching module 128 may send, at step 406, the matching waveforms to the machine learning module 130. Matching waveforms may refer to the standard waveforms that were similar to the input waveform, the waveforms that were generated via convolution and/or cross-correlation, or both. The matching module 128 may return, at step 408, to step 400.

FIG. 5 displays a process for the operation of the machine learning module 130. The process may begin with the machine learning module 130 polling, at step 500, for a set of matching waveforms from the matching module 128. Matching waveforms may be a set of standard waveforms similar to the input waveform or statistical combinations of the input waveform and standard waveforms, such as convolutions or cross-correlations. The machine learning module 130 may input, at step 502, the set of received waveforms into a pre-trained machine learning algorithm. The machine learning algorithm may be pre-trained on similar sets of matched waveforms where the input waveform is from a patient whose health parameters are known. The waveforms may be input directly into the algorithm, such as a set of X and Y values. The matching waveforms may each be summarized as a closest fit function or may be transformed into a set of sine waves using a Fourier transform. The machine learning module 130 may determine, at step 504, if the machine learning algorithm identified any health parameters. Identification may require a certain interval of confidence. For example, if the machine learning algorithm determines that it is more than 70% likely that a health parameter is correct, then that parameter may be considered identified. If there are multiple conflicting parameters, the parameter with the highest confidence may be used. For example, if the algorithm determines that it is 75% likely that the patient's blood glucose level is between 110-115 mg/dL and 90% likely that the patient's blood glucose is between 105-110 mg/dL, then the more confident value of 105-110 mg/dL may be identified. If none of the results from the machine learning algorithm are above the confidence threshold, or the results are otherwise inconclusive, the machine learning module 130 may skip to step 508. If any health parameters were identified, the machine learning module 130 may send, at step 506, the health parameters to the notification module 132. The machine learning module 130 may return, at step 508, to step 500.

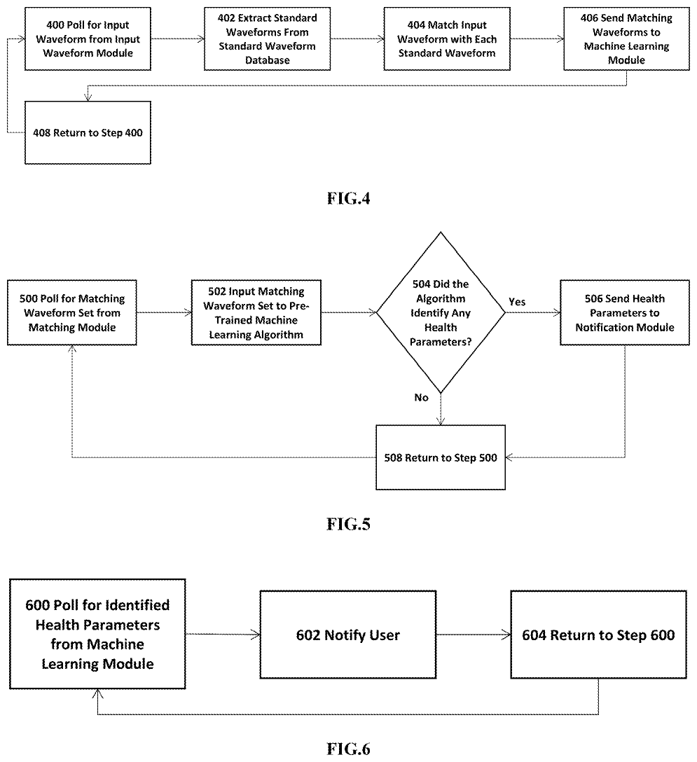

FIG. 6 displays a process for the operation of the notification module 132. The process may begin with the notification module 132 polling, at step 600, for health parameters identified by the machine learning module 130. The notification module 132 may notify, at step 602, the user of the device and/or their care providers. For example, the device may provide the notification at 602 by providing a readable interface with the identified health parameters such as heart rate, blood pressure, blood glucose, oxygen level, etc. This information may be sent via the comms 120 to another device, such as a terminal in a nursing station, doctor's office, emergency medical transport office, etc. Notification may include audio or haptic feedback such as beeping or vibrating. The notification module 132 may return, at step 604, to step 600.

1. A heath parameter monitoring system comprising:
a device that includes one or more transmit antennas configured to transmit radio frequency (RF) waves from one or more transmit antennas into a person over a range of frequencies, and one or more receive antennas that receive return RF waves resulting from the transmitted RF waves into the person and from which a pulse wave signal is generated;
a memory that stores the pulse wave signal;
a standard waveform database stored in the memory;
an input waveform module in communication with the memory that is configured to extracting a segment of the pulse wave signal to generate an extracted segment;
a matching module in communication with the memory and that is configured to receive the extracted segment, compare the extracted segment to the waveforms in the standard waveform database thereby creating matched data, assign a correlation coefficient to each matched data, and determine which correlation coefficients exceed a threshold value;
a machine learning module with a machine learning algorithm that is configured to input into the machine learning algorithm matching waveforms from the matching module for correlation coefficients that exceed the threshold value.

2. The heath parameter monitoring system of claim 1, wherein the matching waveforms comprise the waveforms from the standard waveform database, or waveforms resulting from a convolution and/or cross-correlation of the extracted segment and the waveforms from the standard waveform database.

3. The heath parameter monitoring system of claim 1, wherein each waveform in the standard waveform database includes an associated label.

4. The heath parameter monitoring system of claim 1, wherein the threshold value is user settable or is automatically set.

5. A heath parameter monitoring method comprising:
transmitting radio frequency (RF) waves from one or more transmit antennas into a person over a range of frequencies, and receiving, using one or more receive antennas, a pulse wave signal that results from the RF waves transmitted into the person;
extracting a segment of the pulse wave signal to generate an extracted segment;
comparing the extracted segment to waveforms in a standard waveform database thereby creating matched data;
assigning a correlation coefficient to each matched data, and determining which correlation coefficients exceed a threshold value;
for the correlation coefficients that exceed the threshold value, sending matching waveforms from the correlation coefficients that exceed the threshold value to a machine learning module and inputting the matching waveforms into a machine learning algorithm.

6. The heath parameter monitoring method of claim 5, wherein the matching waveforms comprise the waveforms from the standard waveform database, or waveforms resulting from a convolution and/or cross-correlation of the extracted segment and the waveforms from the standard waveform database.

7. The heath parameter monitoring method of claim 5, wherein each waveform in the standard waveform database includes an associated label.

8. The heath parameter monitoring method of claim 5, wherein the threshold value is user settable or is automatically set.
